(19) United States
(12) Patent Application Publication
(10) Pub. No.: US 2017/0007568 A1
(43) Pub. Date: Jan. 12, 2017

(54) USE OF COMPOSITION CONTAINING IRON (II) AMINO ACID CHELATE IN PREPARATION OF DRUG FOR AMELIORATING DIABETES

(71) Applicant: PROFEAT BIOTECHNOLOGY CO., LTD., Taoyuan County (TW)

(72) Inventors: Tsun-Yuan LIN, Taoyuan County (TW); Hsun-Jin JAN, Taoyuan County (TW); Chai-Hui FU, Taoyuan County (TW); Tsang-Tse CHEN, Taoyuan County (TW); Mu-Kuei CHEN, Taoyuan County (TW); Horng-Mo LEE, Taoyuan County (US)

(21) Appl. No.: 15/121,013

(22) PCT Filed: Nov. 25, 2014
(86) PCT No.: PCT/CN2014/092146

§ 371 Date: Aug. 23, 2016

(51) Int. Cl. A61K 31/295; A61K 9/00

(52) U.S. Cl. CPC A61K 31/295; A61K 9/0053

(57) ABSTRACT

The present invention provides a use of a composition containing a ferrous amino acid chelate for the preparation of a pharmaceutical used in diabetes improvement. The pharmaceutical comprise an effective amount of the composition containing the ferrous amino acid chelate and pharmaceutically acceptable carriers to improve diabetes. The amino acid is able to be chelated with the ferrous iron in a chelated state stably as passing through the stomach. The composition containing the ferrous amino acid chelate can effectively lower blood glucose and improve insulin sensitivity. The pharmaceutical prepared by the composition containing the ferrous amino acid chelate have effect on diabetes improvement.

### BACKGROUND OF THE INVENTION

1. Field of the Invention

The present invention relates to a use of a composition containing a ferrous amino acid chelate, especially to a use of the composition for the preparation of a pharmaceutical used in diabetes improvement.

2. Description of the Prior Art

Metabolic syndrome is a disease of civilization caused by lifestyle and dietary habits of the modern man (Esposito et al., 2007). According to the definition defined by the World Health Organization (WHO) in 1998, a person who has a syndrome of impaired glucose tolerance or insulin resistance, and additional two syndromes of hypertension, obesity, dyslipidemia or microalbuminuria can be diagnosed as suffering the metabolic syndrome. In Taiwan, a person who has three of the following five conditions is diagnosed as having metabolic syndrome. The five conditions include (1) the waist circumference of male is greater than or equal to 90 cm, and the waist circumference of female is greater than or equal to 80 cm; (2) the triacylglycerol is greater than 150 mg/dl; (3) the high-density lipoprotein (HDL) of male is less than 40 mg/dl, and the HDL of female is less than 50 mg/dl; (4) the systolic blood pressure is higher than 130 mmHg, and the diastolic blood pressure is higher than 85 mmHg, and (5) the value of fasting blood glucose is greater than 110 mg/dl. The rate of suffering from metabolic syndrome of Taiwanese is increasing in accordance with increasing age, and among top ten causes of death, many diseases are related to the metabolic syndrome. The average life expectancy of patients with metabolic syndrome is shorter than normal individuals. The reason is that the cardiovascular disease caused by high blood pressure or hyperlipidemia or the diabetes caused by insulin resistance will cause acute complications.

There are two main types of diabetes, type 1 diabetes and type 2 diabetes. The type 1 diabetes is usually diagnosed in young adults or children, and only 5% of people with diabetes suffer type 1 diabetes. A person with the type 1 diabetes cannot produce insulin by himself, and the person needs lifelong insulin therapy. The type 2 diabetes is usually diagnosed in patients aged over 40, and 95% of people with diabetes suffer type 2 diabetes. The type 2 diabetes is caused by impairments of insulin signaling, causing cells to be incapable of glucose uptake. Therefore, patients with type 2 diabetes will keep feeding, which eventually causes high blood pressures. The high concentration of glucose in the blood will stimulate islet cells to secrete insulin and cause hyperinsulinism, and finally generate insulin resistance and cause pancreas failure. Some of the patients with type 2 diabetes need drugs or insulin therapy. Acute complications such as cardiovascular disease (Vlassara, 1996), chronic renal failure (Monnier et al., 1992), retinopathy (Yamagishi et al., 2002), neuropathy and microangiopathy will be caused once the diabetes is not controlled well. Microangiopathy may cause lower limb gangrene and even lead patients to amputation.

Drugs combined with diet control are commonly used as treatment for type 2 diabetes (Granberry and Fonseca, 2005). The common drugs include thiazolidinedione and metformin. Metformin is a kind of oral hypoglycemic drug with biguanide, and the mechanism is unclear. However, it is known that the metformin is able to inhibit hepatic gluconeogenesis and increase glucose uptake in muscle. The side effects caused by the metformin include diarrhea and nausea.

### SUMMARY OF THE INVENTION

For the shortcomings of the side effects caused by treating type 2 diabetes with conventional chemical pharmaceuticals, the objective of the present invention is to provide a use of a composition containing a ferrous amino acid chelate for the preparation of a pharmaceutical used in improvement of diabetes. The composition containing the ferrous amino acid chelate has an effect on improvement of diabetes.

To achieve the above objective, the objective of the invention is to provide the use of a composition containing a ferrous amino acid chelate for preparation of a pharmaceutical used in diabetes improvement, wherein the pharmaceutical comprise an effective amount of the composition containing the ferrous amino acid chelate and pharmaceutically acceptable carriers.

According to the present invention, the term “the composition containing the ferrous amino acid chelate” refers to that the composition is made by mixing an inorganic iron with an amino acid.

Preferably, the inorganic iron includes, but is not limited to, ferrous sulfate, ferrous chloride, or ferrous pyrophosphate, and the amino acid is glycine.

More preferably, the composition containing the ferrous amino acid chelate comprises 95 wt % to 100 wt % of the ferrous glycine chelate. Furthermore preferably, the composition containing the ferrous amino acid chelate comprises 98 wt % to 99.9 wt % of the ferrous glycine chelate.

Preferably, the composition containing the ferrous amino acid chelate is prepared from mixing ferrous sulfate with glycine followed by heating between 60° C. and 90° C. for 8 hours to 48 hours, wherein a weight ratio of the ferrous sulfate to the glycine of ferrous amino acid chelate is between 1:1.2 and 1:1.5.

The composition containing the ferrous amino acid chelate in accordance with the present invention comprises at least one ferrous amino acid chelate, and the chelating ratio of the ferrous iron to the amino acid of the composition containing the ferrous amino acid chelate is between 1:1 and 1:4. More preferably, the chelating ratio of the ferrous iron to the amino acid of ferrous amino acid chelate is between 1:1.5 and 1:2.5.

Preferably, the composition containing the ferrous amino acid chelate comprises a reducing agent. The reducing agent can maintain the oxidation state of the ferrous iron of the ferrous amino acid chelate contained in the composition. Besides, the reducing agent can also enhance the intestinal absorption rate of the composition containing the ferrous amino acid chelate in subjects. The reducing agent includes, but is not limited to, ascorbic acid, citric acid, acetic acid, propionic acid, butyric acid, lactic acid, malic acid, sulfonic acid or succinic acid.

The diabetes improvement in accordance with the present invention means effective treatment or relieving diabetes. As shown in the embodiment of the present invention, the symptoms of diabetes improvement comprise, but are not limited to, lowering blood glucose, improving glucose tolerance, and enhancing insulin sensitivity.

The therapeutically effective amount in accordance with the present invention means dosage of the pharmaceuticals used for therapeutically effective improvement of diabetes in the required period. As shown in the embodiment of the present invention, the dosage of the pharmaceuticals used for effective improvement of diabetes can be determined by administering the composition containing the ferrous amino acid chelate in a specific amount, and measuring the blood glucose, fasting blood glucose and insulin variances in a specific period of time.

Preferably, the therapeutically effective amount of the composition containing the ferrous amino acid chelate is between 0.1 mg/kg/day and 15 mg/kg/day. More preferably, the effective amount is between 0.16 mg/kg/day and 12 mg/kg/day.

The pharmaceutically acceptable carriers in accordance with the present invention comprises any of physiologically compatible solvents, dispersed medium, coating materials, antibacterial agents, antifungal agents, isotonic agents, and absorption delaying agents and analogues thereof. The pharmaceutically acceptable carriers comprise water, saline, phosphate buffered solution, dextrose, glycerol, ethanol, analogues thereof or any combination thereof. In many conditions, preferably, the pharmaceutically acceptable carriers comprise isotonic agents, for example, sugars, polyalcohols such as mannitol and sorbitol, or sodium chloride. The pharmaceutically acceptable carriers can further comprise micro-auxiliary substances such as wetting agent, emulsifier, preservative or buffering agent.

The pharmaceutical in accordance with the present invention have various dosage forms, and the dosage forms include, but are not limited to, liquid, semi-solid and solid. The liquid includes, but is not limited to, dispersion or suspension. The semi-solid and the solid include, but are not limited to, tablet, pill, powder, liposome or suppository. The preferred dosage form of the pharmaceuticals is dependent on the expected mode of administration and therapeutic application.

Preferably, the dosage form of the pharmaceutical in accordance with the present invention is for oral dosage administration or injection. The preferred mode of administration is the mode of enteral administration, such as oral administration. As shown in the embodiment of the present invention, the pharmaceutical comprising the composition containing the ferrous amino acid chelate for effective improvement of diabetes and obesity is orally administered.

Preferably, the pharmaceutical further comprise an excipient, allowing the pharmaceutical to be made in the dosage form applicable to enteral administration or parenteral administration.

Preferably, the dosage form of the pharmaceutical for enteral administration is oral dosage form. The oral dosage form includes, but is not limited to, solution, suspension, tablet or capsule.

The composition containing the ferrous amino acid chelate in accordance with the present invention has greater effect on diabetes improvement than use of commercially available Metformin. Besides, the molecular weight of the amino acid is small enough to be chelated with the ferrous iron in a chelating state stably as passing through the stomach of a subject. The composition containing the ferrous amino acid chelate can effectively lower blood glucose and improve insulin sensitivity.

### BRIEF DESCRIPTION OF THE DRAWINGS

FIG. 1 is a bar chart of fasting blood glucose assay of obese mice fed with high fat diet in accordance with the present invention;

FIG. 2 is a curve chart of oral glucose tolerance assay of diabetic mice fed with a composition A1 in accordance with the present invention, and Met is Metformin; and

FIG. 3 is a bar chart of the area of the curve chart of oral glucose tolerance assay in FIG. 2.

### DETAILED DESCRIPTION OF THE PREFERRED EMBODIMENTS

Preparation 1: Preparation of the Composition Containing a Ferrous Amino Acid Chelate

The method for preparing a composition containing a ferrous amino acid chelate was shown as follows. First, ferrous sulfate was mixed with glycine (above 98% purity) in a weight ratio of 1:1.3 followed by heating from 60° C. to 90° C. for 8 hours to 48 hours to form the composition containing the ferrous amino acid chelate. The chelating ratio of ferrous iron to amino acid of the ferrous amino acid chelate was between 1:1 and 1:4. The composition containing the ferrous amino acid chelate was prepared in concentrations of 0.16 μg/ml, 0.4 μg/ml, 1.2 μg/ml, 4 μg/ml, and 12 μg/ml. The composition containing the ferrous amino acid chelate was named composition A1.

Preparation 2: Animal Study of High Fat Diet-Induced Obesity Mice

TABLE 1

| | Control | Comparison | Experiment | Experiment |
|---|---|---|---|---|
| | group | group | group 1 | group 2 |
| Administered with | — | — | 0.16 mg/kg | 0.4 mg/kg |
| the composition | | | | |
| A1 | | | | |
| Number of mice | 10 | 5 | 5 | 5 |
| Diet | Normal | High fat diet | High fat diet | High fat diet |

C57BL/6JNR male mice at 12 weeks of age (purchased from National Laboratory Animal Center) were fed under 12/12-hour light-dark cycle and supplied with water. The mice were grouped as shown in Table 1. The mice of comparison group, experiment group 1 and experiment group 2 were fed with high fat diet, and the mice of experiment group 1 and experiment group 2 were orally administered with the composition A1 at dosage of 0.16 mg/kg/day and 0.4 mg/kg/day, respectively for 3 months. The weights of the mice were measured every 3 days, and the blood glucose values of the mice were measured every 4 weeks after administering with the composition A1.

### Preparation 3: Animal Study of Drug-Induced Diabetic Mice

C57BL/6JNR male mice at 6 weeks of age (purchased from National Laboratory Animal Center) were fed with normal diet for 1 week. Each of the mice was injected intraperitoneally with 240 mg/kg nicotinamide, and after 15 minutes, each of the mice was injected intraperitoneally with 100 mg/kg streptozotocin. Two days later, each of the mice was injected with nicotinamide and streptozotocin with the same dosages and method as shown above, followed by feeding with the high fat diet (60% fat). Two months later, the mice having fasting blood glucose higher than 140 mg/dl and impaired glucose tolerance (which meant that the blood glucose values were not measured within normal range after injecting glucose for 2 hours) were picked to be type 2 diabetes mice.

The mice were divided into 4 groups which included a control group (supplied with phosphate solution), a metformin (Met) group, a composition A1 with low dose (4 mg/kg/day) group, and a composition A1 with high dose (12 mg/kg/day) group. The weights of the mice were measured every 3 days, and the blood glucose values, oral glucose tolerance assay (OGTT), glycated hemoglobin (HbA1c) and insulin variances of the mice were measured every 4 weeks.

The blood glucose values were measured with a Chemistry Analyzer (Hitachi, Ltd., Japan). The glycated hemoglobin (hemoblobin A1c, HbA1c) was measured with a Glycohemoglobin Analyzer (Tosoh, Ltd., USA). The insulin variance was measured with enzyme-linked immunosorbent assay (ELISA) (Mercodia, Ltd. Sweden).

### EXAMPLE 1

### Examination of Blood Glucose Values, Glucose Tolerance and Insulin Sensitivity of Diabetic Mice Administered with the Composition A1

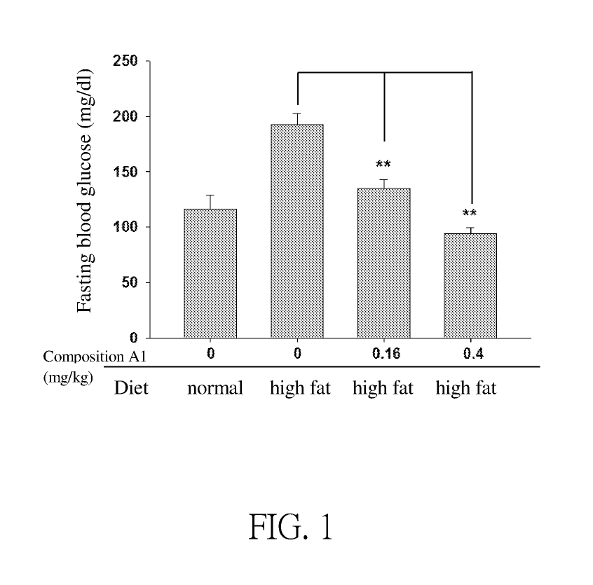

The value of blood glucose was measured according to the method recited in the Preparation 2. With reference to FIG. 1, after administrating for 3 months, results showed that values of the fasting blood glucose of experiment group 1 (administered with 0.16 mg/kg/day composition A1) and experiment group 2 (administered with 0.4 mg/kg/day composition A1) were lower than those of comparison group (without administration with any composition A1) despite that the mice of experiment group 1 and experiment group 2 were fed with high fat diet. The values of the fasting blood glucose of experiment group 2 (administered with 0.4 mg/kg/day composition A1) were lower than those of control group (fed with normal diet, without any high fat diet). Therefore, blood glucose values of the diet-induced obesity mice could be lowered by administering the composition A1.

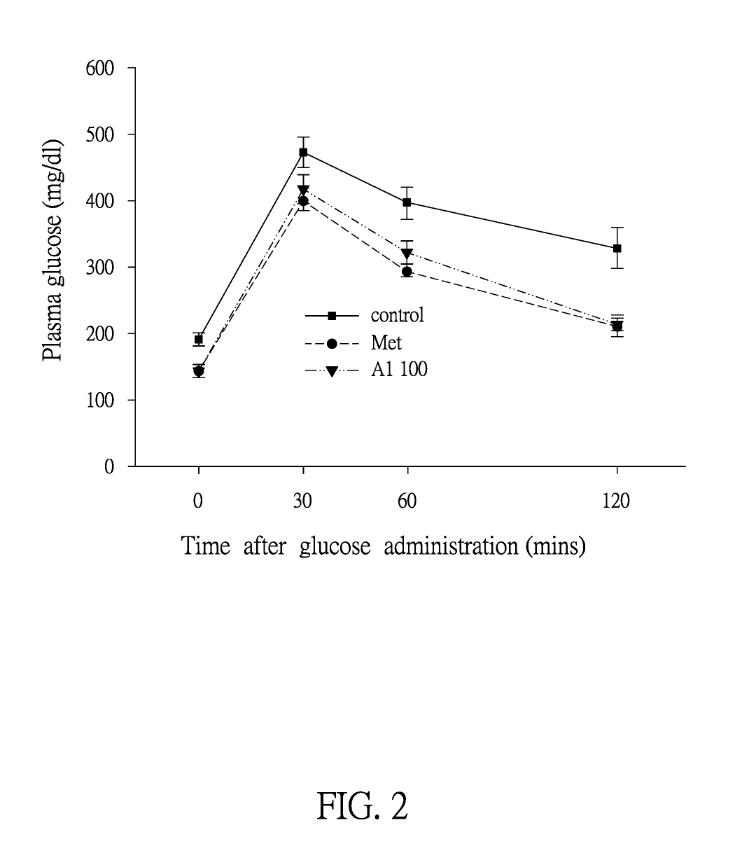

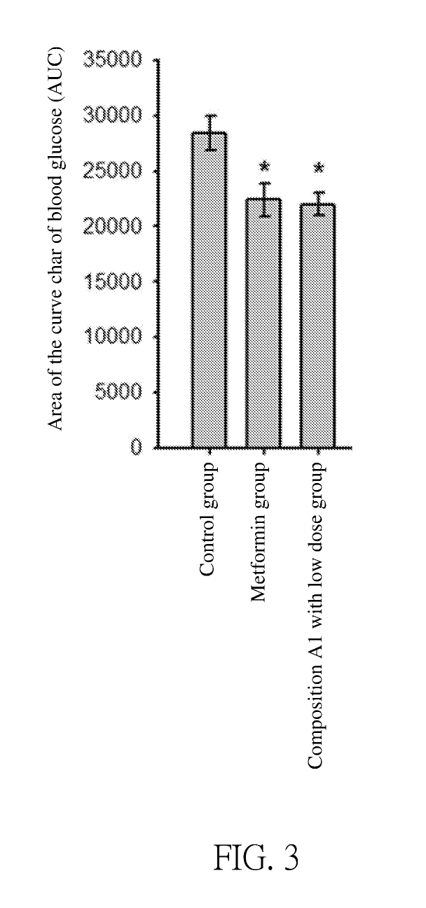

The value of blood glucose was measured according to the method recited in the Preparation 3. With reference to FIG. 2, results showed that glucose tolerances of the metformin group and the composition A1 with high dose group or low dose group were all improved. With reference to FIG. 3, after calculating the area of the curve chart of glucose tolerance assay in FIG. 2, results showed that the composition A1 with low dose could provide greater effect than the metformin with high dose.

TABLE 2
Glycated hemoglobin, values of fasting blood glucose, and insulin
variances of the diabetic mice

| | Glycated hemoglobin | |
|---|---|---|
| | (HbA1c) | insulin |
| Control group | 4.00 ± 0.16 | 18.34 ± 6.68 |
| Metformin group | 3.57 ± 0.04* | 11.11 ± 6.47 |
| Composition A1 with | 3.70 ± 0.03* | 6.00 ± 2.22* |
| low dose group | | |
| Glycine | 3.71 ± 0.03* | 12.47 ± 2.36 |
| Normal value | 2.9 ± 0.05 | |

TABLE 3
Glycated hemoglobin, values of fasting blood glucose, and HOMA-IR
value of the diabetic mice

| | Fasting blood glucose | HOMA-IR |
|---|---|---|
| Control group | 203.00 ± 17.43 | 8.59 ± 2.00 |
| Metformin group | 169.00 ± 10.77* | 4.53 ± 1.42* |
| Composition A1 with | 152.71 ± 4.74* | 2.17 ± 0.87* |
| low dose group | | |
| Composition A1 with | 166.13 ± 5.02* | 3.85 ± 2.08* |
| high dose group | | |
| Glycine | 182.88 ± 11.20 | 5.23 ± 1.73 |
| Normal value | 143.5 ± 9.14 | |

With references to Table 2 and Table 3, after administration of the pharmaceuticals for one month, the value of glycated hemoglobin in each of the groups was decreased obviously. Composition A1 with low dose had the best therapeutic effect on insulin secretion and fasting blood glucose.

The index of homeostasis model assessment for insulin resistance (HOMA-IR) was calculated from the following formula:

HOMA-IR index=insulin (μU/ml)×glucose (mmol/L)/22.5.

Results showed that insulin resistance effects of the metformin group and the composition A1 with low dose group were best, and HOMA-IR value of the composition A1 with low dose group was decreased from 8.59 to 2.17, which was better than 4.53 of the metformin group. Therefore, the composition A1 with low dose group had greater effects on fasting blood glucose, insulin, and HOMA-IR than the metformin group. Besides, the dosage of the composition A1 group was lower than that of the metformin group. Therefore, the composition A1 with low dose had the best effect on improvement of diabetes.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A use of a composition comprising a ferrous amino acid chelate for the preparation of a pharmaceutical used in diabetes improvement, wherein the pharmaceuticals comprise an effective amount of the composition containing the ferrous amino acid chelate, and pharmaceutically acceptable carriers.

2. The use as claimed in claim 1, wherein the chelating ratio of ferrous iron to amino acid of the composition containing the ferrous amino acid chelate is between 1:1 and 1:4.

3. The use as claimed in claim 1, wherein the chelating ratio of ferrous iron to amino acid of the composition containing the ferrous amino acid chelate is between 1:1.5 and 1:2.5.

4. The use as claimed in claim 1, wherein the effective amount of the composition containing the ferrous amino acid chelate is between 0.1 mg/kg/day and 15 mg/kg/day.

5. The use as claimed in claim 1, wherein the effective amount of the composition containing the ferrous amino acid chelate is between 0.16 mg/kg/day and 12 mg/kg/day.

6. The use as claimed in claim 1, wherein the composition containing the ferrous amino acid chelate is prepared from mixing an inorganic iron with an amino acid followed by heating between 60° C. and 90° C. for 8 hours to 48 hours, wherein the chelating ratio of the inorganic iron to the amino acid is between 1:1.2 and 1:1.5.

7. The use as claimed in claim 6, wherein the inorganic iron is ferrous sulfate, ferrous chloride, or ferrous pyrophosphate, wherein the amino acid is glycine.

8. The use as claimed in claim 6, wherein the composition containing the ferrous amino acid chelate comprises a reducing agent, wherein the reducing agent comprises ascorbic acid, citric acid, acetic acid, propionic acid, butyric acid, lactic acid, malic acid, sulfonic acid or succinic acid.

9. The use as claimed in claim 1, wherein a dosage form of the pharmaceutical is for enteral administration or parenteral administration.

10. The use as claimed in claim 9, wherein the dosage form of the pharmaceutical for enteral administration is oral dosage form, wherein the oral dosage form comprises solution, suspension, tablet or capsule.
